SEQUENCE LISTING (17 sequences)

SEQ ID NO: 1 - DNA, 1353 bp, Artificial Sequence
  DNA encoding fusion protein of Lipoprotein D fragment, Mage3 fragment, and histidine tail
atggatccaaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggatctggaacagcgtagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgaattggttcattttctgctcctcaagtatcgagccagggagccggtcacaaaggcagaaatgctggggagtgtcgtcggaaattggcagtattttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagggcggtcatcaccatcaccatgagtgggttttgagagagggggaagagggcggtcatcaccatcaccatcaccattaa

SEQ ID NO: 2 - protein, 450 aa, Artificial Sequence
  Synthetic construct
MDPKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMDLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEGGHHHHHHH

SEQ ID NO: 3 - DNA, 1341 bp, Artificial Sequence
  DNA encoding fusion protein of Lipoprotein D fragment, MAGE1 fragment, and histidine tail
atggatccaaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatgggctctctggaacagcgtagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtcggcggtcatttcttcccatccctgcgtgaagcagctttgagagaggaggaagagggagtcggcggtcatcaccatcaccatcaccattaa

SEQ ID NO: 4 - protein, 446 aa, Artificial Sequence [sequence omitted]

SEQ ID NO: 5 - protein, 403 aa, Artificial Sequence [sequence omitted]

SEQ ID NO: 6 - DNA, 1212 bp, Artificial Sequence
  DNA coding fusion protein NS1-MAGE3-His
atggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcactcttggtctggacatcgagacagccacacgtgctggaaagcagatagtggagcggattctgaaagaagaatccgatgaggcacttaaaatgaccatggatctggaacagcgtagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgaattggttcattttctgctcctcaagtatcgagccagggagccggtcacaaaggcagaaatgctggggagtgtcgtcggaaattggcagtatttctttcctgtgatcttcagcaaagcttccagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagggcggtcatcaccatcaccatcaccattaa

SEQ ID NO: 7 - protein, 445 aa, Artificial Sequence
  Fusion protein of CLYTA-MAGE1-Histidine
MKGGIVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPDGTLADRPELDMGSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGVGGHHHHHHH

SEQ ID NO: 8 - DNA, 1338 bp, Artificial Sequence
  DNA coding fusion protein CLTA-MAGE1-His
atgaaagggggaattgtacattcagacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatggtatcaaatgcctttatccagtcagcggacggaacaggctggtactacctcaaaccagacggaacactggcagacaggccagaattggacatgggctctctggaacagcgtagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtcggcggtcatcaccatcaccatcaccattaa

SEQ ID NO: 9 - protein, 453 aa, Artificial Sequence
  Fusion protein CLTA-MAGE1-Histidine
MKGGIVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPDGTLADRPELASMLDMDLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEGGHHHHHHH

SEQ ID NO: 10 - DNA, 1362 bp, Artificial Sequence
  DNA for fusion protein CLYTA-MAGE3-His
atgaaagggggaattgtacattcagacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatggtatcaaatgcctttatccagtcagcggacggaacaggctggtactacctcaaaccagacggaacactggcagacaggccagaattggccagcatgctggacatggatctggaacagcgtagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtctgagttccaagcagcactcagtaggaaggtggccaagttggttcattttctgctcctcaagtatcgagccagggagccggtcacaaaggcagaaatgctggggagtgtcgtcggaaattggcagtacttctttcctgtgatcttcagcaaagcttccgattccttgcagctggtctttggcatcgagctgatggaagtggaccccatcggccacgtgtacatctttgccacctgcctgggcctctcctacgatggcctgctgggtgacaatcagatcatgcccaagacaggcttcctgataatcatcctggccataatcgcaaaagagggcgactgtgcccctgaggagaaaatctgggaggagctgagtgtgttagaggtgtttgaggggagggaagacagtatcttcggggatcccaagaagctgctcacccaatatttcgtgcaggaaaactacctggagtaccggcaggtccccggcagtgatcctgcatgctatgagttcctgtggggtccaagggccctcattgaaaccagctatgtgaaagtcctgcaccatatggtaaagatcagtggaggacctcgcatttcctacccactcctgcatgagtgggctttgagagagggggaagagggcggtcatcaccatcaccatcaccattaa

SEQ ID NO: 11 - protein, 309 aa, Artificial Sequence
  309 Amino Acid Sequence of the MAGE A1 protein
MSLEQRSLHCKPEEALEAQQEALGLVCVQAAASSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 12 - DNA, 20 bp, Artificial Sequence
  CpG containg oligonucleotide  (CpG 1826)
tccatgacgttcctgacgtt

SEQ ID NO: 13 - DNA, 18 bp, Artificial Sequence
  CpG containing oligonucleotide  (CpG 1758)
tctcccagcgtgcgccat

SEQ ID NO: 14 - DNA, 30 bp, Artificial Sequence
  CpG containing oligonucleotide
accgatgacgtcgccggtgacggcaccacg

SEQ ID NO: 15 - DNA, 24 bp, Artificial Sequence
  CpG containing oligonucleotide (CpG 2006)
tcgtcgttttgtcgttttgtcgtt

SEQ ID NO: 16 - DNA, 20 bp, Artificial Sequence
  CpG containing oligonucleotide (CpG 1668)
tccatgacgttcctgatgct

SEQ ID NO: 17 - protein, 85 aa, Artificial Sequence
  MAGE Core signature sequence - consensus
LIXVLXXIXXXGXXAPEEXIWEXLXXMXXXXGXEXXXXGXPXXLLTXXXVQEXYLXYXQVPXSXPXXYEFLWGPRAXXETXXXKV

(19) United States
(12) Patent Application Publication
(10) Pub. No.: US 2009/0186049 A1
(43) Pub. Date: Jul. 23, 2009

(54) METHOD OF TREATING MAGE POSITIVE CANCER

(76) Inventors: Vincent Brichard, Rixensart (BE); Frederic Eugene Lehmann, Rixensart (BE)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/301,541

(22) PCT Filed: May 24, 2007
(86) PCT No.: PCT/EP07/55037

§ 371 Date: Nov. 19, 2008

(30) Foreign Application Priority Data
May 26, 2006 (GB) 0610547.2
Apr. 16, 2007 (GB) 0707307.5

(51) Int. Cl. A61K 39/145; A61K 39/09; A61K 39/00

(52) U.S. Cl. 424/209.1; 424/244.1; 424/277.1

(57) ABSTRACT

The present invention relates to methods for treating a range of cancers, for example MAGE positive cancers, including, but not limited to melanoma and non-small cell lung cancer (NSCLC). The present invention relates to methods for treating cancer in the adjuvant (eg. post-operative) setting and the active disease setting.

### FIELD OF THE INVENTION

The present invention relates to methods for treating a range of MAGE-expressing cancers, including, but not limited to: melanoma; breast cancer; liver cancer; bladder cancer including transitional cell carcinoma; lung cancer including non-small cell lung cancer (NSCLC); head and neck cancer including: squamous cell carcinoma and oesophagus carcinoma; colon carcinoma; seminoma; and multiple myeloma. The present invention relates to methods for treating MAGE-expressing cancers in the adjuvant (eg. post-operative) setting and in active disease, and to the use of a vaccine in the treatment of patients suffering from MAGE-expressing cancers in these settings.

### BACKGROUND TO THE INVENTION

### MAGE Antigens

MAGE antigens are antigens encoded by the family of Melanoma-associated antigen genes (MAGE). MAGE genes are predominately expressed on melanoma cells (including malignant melanoma) and some other cancers including: NSCLC (non small cell lung cancer), head and neck cancer, including: squamous cell carcinoma; and oesophagus carcinoma; bladder cancer, including: transitional cell carcinoma; but are not detectable on normal tissues except in the testis and the placenta (Gaugler, 1994; Weynants, 1994; Patard, 1995). MAGE-3 is expressed in 69% of melanomas (Gaugler, 1994), and can also be detected in 44% of NSCLC (Yoshimatsu 1988), 48% of head and neck squamous cell carcinoma, 34% of bladder transitional cell carcinoma 57% of oesophagus carcinoma 32% of colon cancers and 24% of breast cancers (Van Pel, 1995); Inoue, 1995 Fujie 1997; Nishimura 1997). Cancers expressing MAGE proteins are known as MAGE associated tumours.

The MAGE-1 gene belongs to a family of 12 closely related genes, MAGE 1, MAGE 2, MAGE 3, MAGE 4, MAGE 5, MAGE 6, MAGE 7, MAGE 8, MAGE 9, MAGE 10, MAGE 11, MAGE 12, located on chromosome X and sharing with each other 64 to 85% homology in their coding sequence (De Plaen, 1994). These are sometimes known as MAGE A1, MAGE A2, MAGE A3, MAGE A4, MAGE A5, MAGE A6, MAGE A7, MAGE A8, MAGE A9, MAGE A 10, MAGE A11, MAGE A 12 (The MAGE A family).

Two other groups of proteins are also part of the MAGE family although more distantly related. These are the MAGE B and MAGE C group. The MAGE B family includes MAGE B1 (also known as MAGE Xp1, and DAM 10), MAGE B2 (also known as MAGE Xp2 and DAM 6) MAGE B3 and MAGE B4—the Mage C family currently includes MAGE C1 and MAGE C2.

### BRIEF DESCRIPTION OF DRAWINGS

FIG. 1 provides the nucleotide and encoded amino acid sequence of a fusion protein of a Lipoprotein D fragment, a MAGE A3 fragment, and a histidine tail (SEQ ID Nos: 1 and 2).

FIG. 2 provides the nucleotide sequence encoding a fusion protein of a Lipoprotein D Fragment, a MAGE A1 fragment, and a histidine tail (SEQ ID NO:3)

FIG. 3 provides the amino acid sequence of a fusion protein of Lipoprotein D, MAGE A1, and a histidine tail (SEQ ID NO:4).

FIG. 4 provides the amino acid sequence of a fusion protein of NS-1, MAGE A3, and a histidine tail (SEQ ID NO:5).

FIG. 5 provides the nucleotide sequence of a fusion protein of NS-1, MAGE A3, and a histidine tail (SEQ ID NO:6).

FIG. 6 provides the amino acid sequence of a fusion protein of CLYTA, MAGE A1, and a histidine tail (SEQ ID NO:7).

FIG. 7 provides the nucleotide sequence encoding a fusion protein of NS-1, MAGE A1, and a histidine tail (SEQ ID NO:8).

FIG. 8 provides the amino acid sequence of a fusion protein of CLYTA, MAGE A3, and a histidine tail (SEQ ID NO:9).

FIG. 9 provides the nucleotide sequence encoding a fusion protein of CLYTA, MAGE A3, and a histidine tail (SEQ ID NO:10).

FIG. 10 provides the 309 amino acid MAGE A1 protein (SEQ ID NO:11, see also GenBank protein Sequence Reference No. 004979), the core signature corresponds to amino acid 195-279.

### DETAILED DESCRIPTION

In general terms, a MAGE A protein can be defined as containing a core sequence signature located towards the C-terminal end of the protein. For example with respect to the 309 amino acid MAGE A1 protein (SEQ ID NO:11, see also GenBank protein Sequence Reference No. 004979), the core signature corresponds to amino acid 195-279.

The consensus pattern of the core signature is thus described as follows wherein x represents any amino acid, lower case residues are conserved (conservative variants allowed) and upper case residues are perfectly conserved.

### Core Sequence Signature

LixvL(2x)I(3x)g(2x)apEExiWex1(2x)m(3-4x)Gxe(3-4x)gxp(2x)11t(3x)VqexYLxYxqVPxsxP(2x)yeFLWGprA(2x)Et(3x)kv

Conservative substitutions are well known and are generally set up as the default scoring matrices in sequence alignment computer programs. These programs include PAM250 (Dayhoft M. O. et al., (1978), “A model of evolutionary changes in proteins”, In “Atlas of Protein sequence and structure” 5(3) M. O. Dayhoft (ed.), 345-352), National Biomedical Research Foundation, Washington, and Blosum 62 (Steven Henikoft and Jorja G. Henikoft (1992), “Amino acid substitution matricies from protein blocks”), Proc. Natl. Acad. Sci. USA 89 (Biochemistry): 10915-10919.

In general terms, substitution within the following groups are conservative substitutions, but substitutions between groups are considered non-conserved. The groups are:

### i) Aspartate/asparagine/glutamate/glutamine

### ii) Serine/threonine

iii) Lysine/arginine

### iv) Phenylalanine/tyrosine/tryptophane

### v) Leucine/isoleucine/valine/methionine

### vi) Glycine/alanine

In general and in the context of this invention, a MAGE protein will be approximately 50% identical in this core region with amino acids 195 to 279 of MAGE A1 (SEQ ID NO:11).

MAGE protein derivatives are also known in the art, see: WO99/40188. Such derivatives are suitable for use in therapeutic vaccine formulations which are suitable for the treatment of a range of tumour types.

### Cancer Types

This invention may be used for patients having MAGE-expressing cancers, such as: melanoma; breast cancer; bladder cancer, including transitional cell carcinoma; lung cancer including NSCLC; head and neck cancer including squamous cell carcinoma; and oesophagus carcinoma; colon carcinoma; multiple myeloma. In an embodiment, the invention may be used in an adjuvant (eg. post surgical removal of the primary tumor) setting in such cancers. In another embodiment, the invention may be used in an active disease setting. The invention may also be used in patients having cancers expressing other tumour antigens, provided that the cancer immunotherapy is antigen-specific cancer immunotherapy, ie that the tumour expresses at least one of the antigens on which the immunotherapy is based.

### Lung Cancer

There are two types of lung cancer: non-small cell lung cancer (NSCLC) and small cell lung cancer (SCLC). The names simply describe the type of cell found in the tumours. NSCLC includes squamous-cell carcinoma, adenocarcinoma, and large-cell carcinoma and accounts for around 80% of lung cancers. NSCLC is hard to cure and treatments available tend to have the aim of prolonging life as far as possible and relieving symptoms of disease.

About half of all patients with completely resected early-stage NSCLC will relapse.

Patients in this adjuvant setting (eg. post-surgical removal of the primary tumor) are defined as those in which the primary tumor has been surgically resected, or those patients considered to be free or substantially free of detectable cancer tissue (primary and/or metastatic). Currently, while adjuvant-setting chemotherapy may reduce the number of relapses, this may be associated with substantial toxicity.

Throughout this specification and the appended claims, unless the context requires otherwise, the words “comprise” and “include” or variations such as “comprising”, “comprises”, “including”, “includes”, etc., are to be construed inclusively, that is, use of these words will imply the possible inclusion of integers or elements not specifically recited.

As used herein, “Swiss-style” or “use” wording may replace “method of treatment” wording, as required.

All publications, including but not limited to patents and patent applications, cited in this specification are herein incorporated by reference as if each individual publication were specifically and individually indicated to be incorporated by reference herein as though fully set forth.

### Sequences

SEQ ID NO: 1—nucleotide of a fusion protein of a Lipoprotein D fragment, a MAGE A3 fragment, and a histidine tail
SEQ ID NO:2—amino acid sequence of a fusion protein of a Lipoprotein D fragment, a MAGE A3 fragment, and a histidine tail.
SEQ ID NO:3—nucleotide sequence encoding a fusion protein of a Lipoprotein D Fragment, a MAGE A1 fragment, and a histidine tail.
SEQ ID NO:4—amino acid sequence of a fusion protein of Lipoprotein D, MAGE A1, and a histidine tail.
SEQ ID NO:5—amino acid sequence of a fusion protein of NS-1, MAGE A3, and a histidine tail.
SEQ ID NO:6—nucleotide sequence of a fusion protein of NS-1, MAGE A3, and a histidine tail.
SEQ ID NO:7: amino acid sequence of a fusion protein of CLYTA, MAGE A1, and a histidine tail.
SEQ ID NO:8—nucleotide sequence encoding a fusion protein of NS-1, MAGE A1, and a histidine tail.
SEQ ID NO:9—amino acid sequence of a fusion protein of CLYTA, MAGE A3, and a histidine tail.
SEQ ID NO: 10—nucleotide sequence encoding a fusion protein of CLYTA, MAGE A3, and a histidine tail.
SEQ ID NO: 11—309 amino acid sequence of MAGE A1.
SEQ ID NO:12—CpG containing oligonucleotide (CpG1826).
SEQ ID NO:13 CpG containing oligonucleotide (CpG1758)
SEQ ID NO:14 CpG containing oligonucleotide
SEQ ID NO:15 CpG containing oligonucleotide (CpG2006)
SEQ ID NO:16—CpG containing oligonucleotide (CpG1668)

### STATEMENT OF THE INVENTION

In one embodiment of the present invention, there is provided a method of treating a patient having cancer, for example a MAGE-expressing cancer, such as melanoma; breast cancer; bladder cancer, including transitional cell carcinoma; lung cancer including NSCLC; head and neck cancer including squamous cell carcinoma, and oesophagus carcinoma; colon carcinoma; multiple myeloma, in an adjuvant (eg. post-operative) setting, comprising administering to the patient a composition comprising a tumour associated antigen, for example an antigen from the MAGE protein family, or a nucleotide sequence encoding the tumour associated antigen.

The MAGE protein may comprise a MAGE-A protein having at least the core MAGE signature, that corresponds to amino acids 195-279 of MAGE-A1.

In one embodiment, the MAGE protein is selected from the group MAGE A1, MAGE A2, MAGE A3, MAGE A4, MAGE A5, MAGE A6, MAGE A7, MAGE A8, MAGE A9, MAGE A10, MAGE A11, MAGE A12, MAGE B1, MAGE B2, MAGE B3 and MAGE B4, MAGE C1, MAGE C2. In one embodiment, the MAGE protein is selected from MAGE-A1 or MAGE-A3.

In one embodiment of the present invention, therapy with antigens other than (or in addition to) MAGE may be considered, for treatment of tumours expressing other antigens. Exemplary antigens or derivatives or fragments include the cancer testes antigens PRAME (WO 96/10577), BAGE, RAGE, LAGE 1 (WO 98/32855), LAGE 2 (also known as NY-ESO-1, WO 98/14464), XAGE (Liu et al, Cancer Res, 2000, 60:4752-4755; WO 02/18584) SAGE, and HAGE (WO 99/53061) or GAGE (Robbins and Kawakami, 1996, Current Opinions in Immunology 8, pps 628-636; Van den Eynde et al., International Journal of Clinical & Laboratory Research (1997); Correale et al. (1997), Journal of the National Cancer Institute 89, p293. Indeed these antigens are expressed in a wide range of tumour types such as melanoma, lung carcinoma, sarcoma and bladder carcinoma.

As used herein, the term “positive” for example MAGE-positive, is interchangeable with the term “expressing”, for example MAGE-expressing, in the context of whether an antigen is expressed by a cell.

In one embodiment, the antigen for use in the present invention may comprise or consist of WT-1 expressed by the Wilm's tumor gene, or its N-terminal fragment WT-1F comprising about or approximately amino acids 1-249

In one embodiment prostate antigens may be utilised, such as prostate cancer antigens or prostate specific differentiation antigen (PSA), PAP, PSCA (PNAS 95(4) 1735-1740 1998), PSMA or the antigen known as prostase.

In one embodiment, the prostate antigen is P501S or a fragment thereof. P501S, also named prostein (Xu et al., Cancer Res. 61, 2001, 1563-1568), is a 553 amino acid protein (SEQ ID NO. 113 of WO98/37814). Immunogenic fragments and portions thereof comprising at least 20, at least 50, or at least 100 contiguous amino acids are disclosed in the above referenced patent application and may be used as antigens in the present invention. Fragments are disclosed in WO 98/50567 (PS108 antigen) and as prostate cancer-associated protein (SEQ ID NO: 9 of WO 99/67384). Other fragments are amino acids 51-553, 34-553 or 55-553 of the full-length P501S protein.

Prostase is a prostate-specific serine protease (trypsin-like), 254 amino acid-long, with a conserved serine protease catalytic triad H-D-S and a amino-terminal pre-propeptide sequence, indicating a potential secretory function (P. Nelson, Lu Gan, C. Ferguson, P. Moss, R. Linas, L. Hood & K. Wand, “Molecular cloning and characterisation of prostase, an androgen-regulated serine protease with prostate restricted expression, In Proc. Natl. Acad. Sci. USA (1999) 96, 3114-3119). A putative glycosylation site has been described. The predicted structure is very similar to other known serine proteases, showing that the mature polypeptide folds into a single domain. The mature protein is 224 amino acids-long, with at least one A2 epitope shown to be naturally processed. Prostase nucleotide sequence and deduced polypeptide sequence and homologous are disclosed in Ferguson, et al. (Proc. Natl. Acad. Sci. USA 1999, 96, 3114-3119) and in International Patent Applications No. WO 98/12302 (and also the corresponding granted patent U.S. Pat. No. 5,955,306), WO 98/20117 (and also the corresponding granted patents U.S. Pat. No. 5,840,871 and U.S. Pat. No. 5,786,148) (prostate-specific kallikrein) and WO 00/04149 (P703P).

Other prostate specific antigens that may be used in the present invention are known from WO98/37418, and WO/004149. Another is STEAP(PNAS 96 14523 14528 7-12 1999).

Other tumour associated antigens useful in the context of the present invention include: Plu-1 J. Biol. Chem. 274 (22) 15633-15645, 1999, HASH-1, HASH-2 (Alders, M. et al., Hum. Mol. Genet. 1997, 6, 859-867), Cripto (Salomon et al Bioessays 199, 21 61-70,U.S. Pat. No. 5,654,140), CASB616 (WO 00/53216), Criptin (U.S. Pat. No. 5,981,215). Further antigens that may be used include tyrosinase, telomerase, P53, NY-Br1.1 (WO 01/47959) and fragments thereof such as disclosed in WO 00/43420, B726 (WO 00/60076, SEQ ID nos 469 and 463; WO 01/79286, SEQ ID nos 474 and 475), P510 (WO 01/34802 SEQ ID nos 537 and 538) and survivin.

Other antigens that may be used include breast cancer antigens such as Her-2/neu, mammaglobin (U.S. Pat. No. 5,668,267), B305D (WO00/61753 SEQ ID nos 299, 304, 305 and 315), or those disclosed in WO00/52165, WO99/33869, WO99/19479, WO 98/45328. Her-2/neu antigens are disclosed inter alia, in U.S. Pat. No. 5,801,005. In one embodiment, the antigen may comprise the entire extracellular domain of Her-2/neu (comprising approximately amino acid 1-645) or fragments thereof and at least an immunogenic portion of or the entire intracellular domain approximately the C terminal 580 amino acids. In particular, the intracellular portion may comprise the phosphorylation domain or fragments thereof. Such constructs are disclosed in WO00/44899. One construct of Her-2/neu that may be used is known as ECD-PD, a second is known as ECD deltaPD (see WO00/44899) also named dHer2. The Her-2/neu as used herein can be derived from rat, mouse or human.

In one embodiment the tumour antigen may be a small peptide antigen (ie less than about 50 amino acids). Exemplary peptides include Mucin-derived peptides such as MUC-1 (see for example U.S. Pat. No. 5,744,144; U.S. Pat. No. 5,827,666; WO88/05054, U.S. Pat. No. 4,963,484). Specifically contemplated are MUC-1 derived peptides that comprise at least one repeat unit of the MUC-1 peptide, preferably at least two such repeats and which is recognised by the SM3 antibody (U.S. Pat. No. 6,054,438). Other mucin derived peptides include peptides from MUC-5.

The tumour antigen for use in the present invention may be full length native protein or a chemically or genetically modified derivative thereof. Alternatively, immunogenic fragments of the protein, for example comprising 9 to 20 such as 9 to 100 amino acids may be employed.

In one embodiment, the antigen may comprise a tumour antigen as described herein linked to an immunological fusion or expression enhancer partner.

The antigen and partner may be chemically conjugated, or may be expressed as a recombinant fusion protein. In an embodiment in which the antigen and partner are expressed as a recombinant fusion protein, this may allow increased levels to be produced in an expression system compared to non-fused protein. Thus the fusion partner may assist in providing T helper epitopes (immunological fusion partner), preferably T helper epitopes recognised by humans, and/or assist in expressing the protein (expression enhancer) at higher yields than the native recombinant protein. In one embodiment, the fusion partner may be both an immunological fusion partner and expression enhancing partner.

In one embodiment of the invention, the immunological fusion partner that may be used is derived from protein D, a surface protein of the gram-negative bacterium, Haemophilus influenza B (WO91/18926) or a derivative thereof. The protein D derivative may comprise the first ⅓ of the protein, or approximately or about the first ⅓ of the protein, in particular it may comprise the first N-terminal 100-110 amino acids or approximately the first N-terminal 100-110 amino acids. In one embodiment, the protein D or derivative thereof may be lipidated. Generally the protein D derivative comprises approximately or about the first ⅓ of the protein, in particular approximately the first N-terminal 100-120 amino acids such as the first 109 to 112 amino acids, more specifically the first 109 amino acids (or 108 amino acids thereof).

The first 109 residues of the Lipoprotein D fusion partner may provide the vaccine candidate antigen with additional exogenous T-cell epitopes and increase expression level in E-coli (thus acting also as an expression enhancer). The lipid tail may ensure optimal presentation of the antigen to antigen presenting cells.

In one embodiment, the fusion partner may comprise or consist of the first third of protein D without its signal sequence, for example the fusion partner may comprise amino acids 20 to 127 or a sequence comprising or consisting of about amino acid 20 to about amino acid 127). In one aspect the invention provides a fusion protein wherein the N-terminal portion of protein D (as described above) is fused to the N-terminus of the cancer testis antigen or an immunogenic fragment thereof. More specifically the fusion with the protein D and the N-terminus of the tumour antigen is effected such that the cancer testis antigen replaces the C-terminal-fragment of protein D that has been excised. Thus the N-terminus of protein D becomes the N-terminus of the fusion protein.

Fusion proteins of the invention may include an affinity tag, such as for example, a histidine tail comprising between 5 to 9 such as 6 histidine residues. These residues may, for example be on the terminal portion of protein D (such as the N-terminal of protein D) and/or the may be fused to the terminal portion of the tumour antigen. Generally however the histidine tail with be located on terminal portion of the cancer testis antigen such as the C-terminal end of the tumour antigen. Histidine tails may be advantageous in aiding purification.

The composition of the present invention may comprise a mixture of one or more tumour antigens as described herein, and/or one or more peptides thereof, and/or or more fusion proteins thereof.

Alternatively, vectors comprising DNA encoding for the protein or an immunogenic fragment thereof may be administered.

An immune response may generated against the vector carrying the encoding DNA and thus the general immune response may be boosted (ie the vector is itself acting as an adjuvant).

The immunotherapy may, for example be administered in a prime boost regime.

Other fusion partners that may be used include the non-structural protein from influenzae virus, NS1 (hemagglutinin). Typically the N terminal 81 amino acids of NS1 may be utilised, although different fragments may be used provided they include T-helper epitopes.

In another embodiment the immunological fusion partner is the protein known as LytA. LytA is derived from Streptococcus pneumoniae which synthesise an N-acetyl-L-alanine amidase, amidase LytA, (coded by the LytA gene {Gene, 43 (1986) page 265-272} an autolysin that specifically degrades certain bonds in the peptidoglycan backbone. The C-terminal domain of the LytA protein is responsible for the affinity to the choline or to some choline analogues such as DEAE. This property has been exploited for the development of E. coli C-LytA expressing plasmids useful for expression of fusion proteins. Purification of hybrid proteins containing the C-LytA fragment at its amino terminus has been described {Biotechnology: 10, (1992) page 795-798}. In one embodiment, the C terminal portion of the molecule may be used. The embodiment may utilise the repeat portion of the LytA molecule found in the C terminal end starting at residue 178. In one embodiment, the LytA portion may incorporate residues 188-305.

In one embodiment of the present invention, the MAGE protein may comprise a derivatised free thiol. Such antigens have been described in WO99/40188. In particular carboxyamidated or carboxymethylated derivatives may be used.

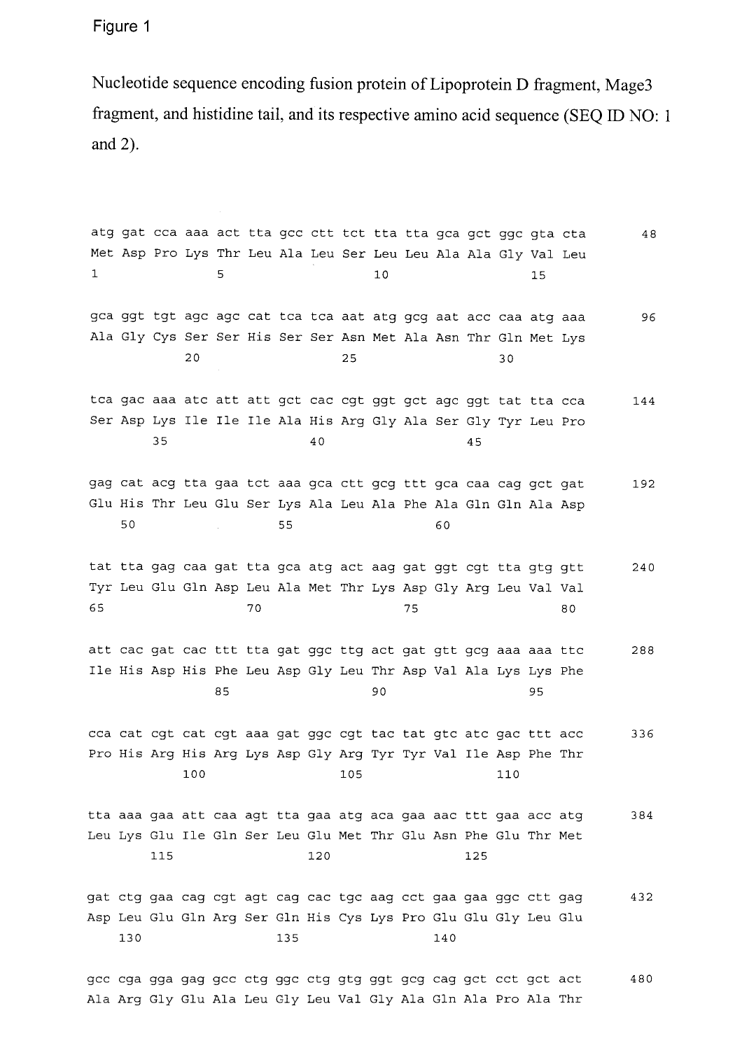

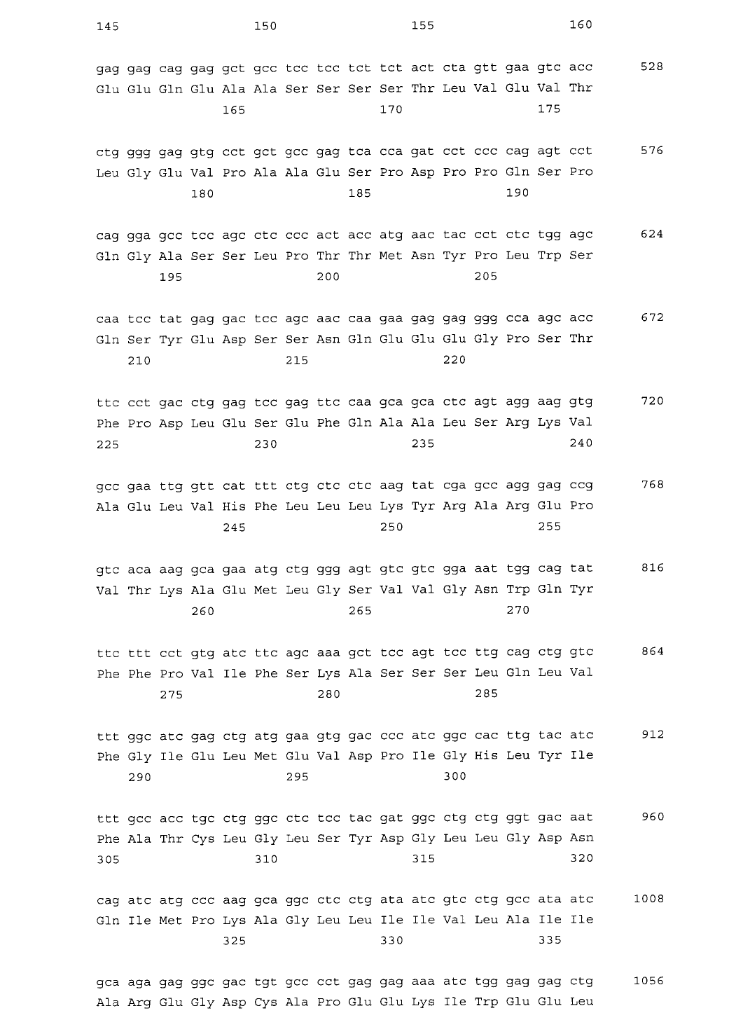

In one embodiment of the present invention, the antigen comprises a MAGE-A3-Protein D molecule. The nucleotide and amino acid sequences for this molecule are shown in FIG. 1 (SEQ ID NO: 1 and 2, respectively). This antigen and those summarised below are described in more detail in WO99/40188.

In further embodiments of the present invention, the tumour associated antigen may comprise a MAGE antigen as described herein, in any of the following fusion proteins:

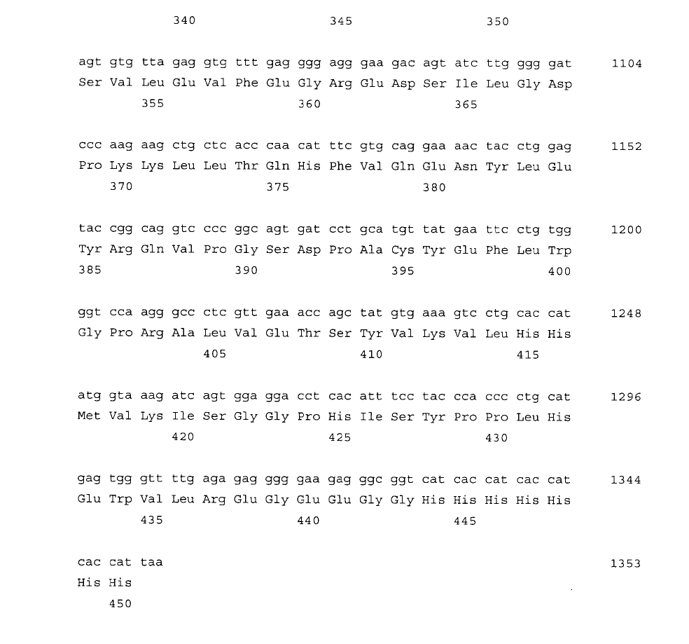

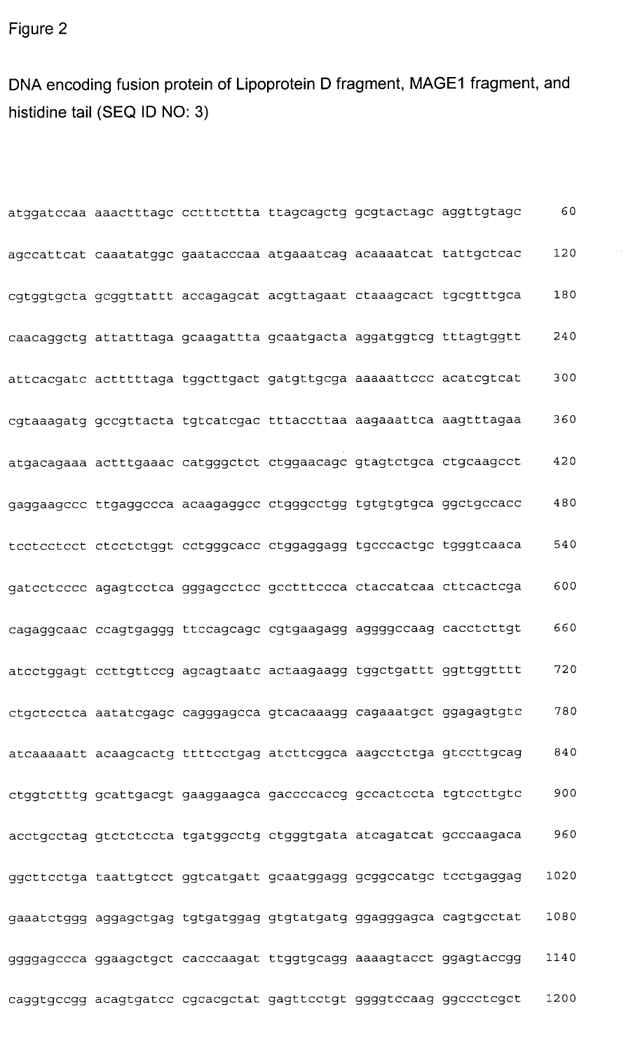

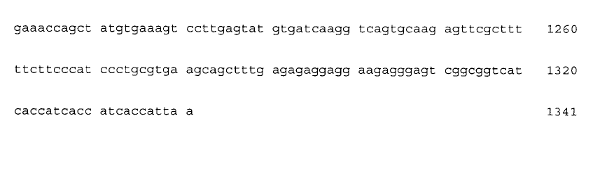

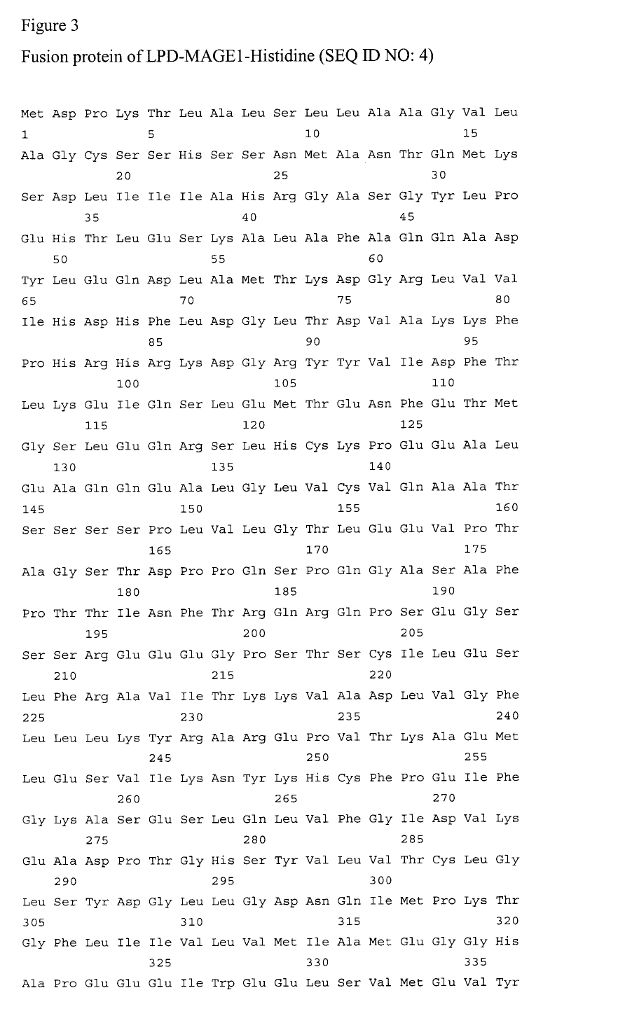

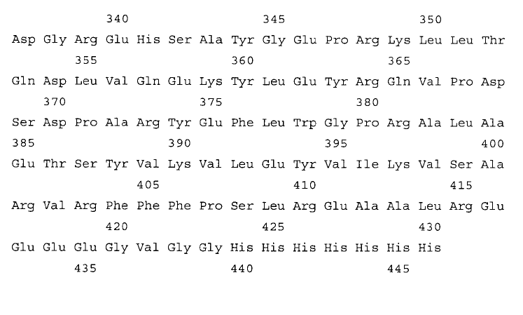

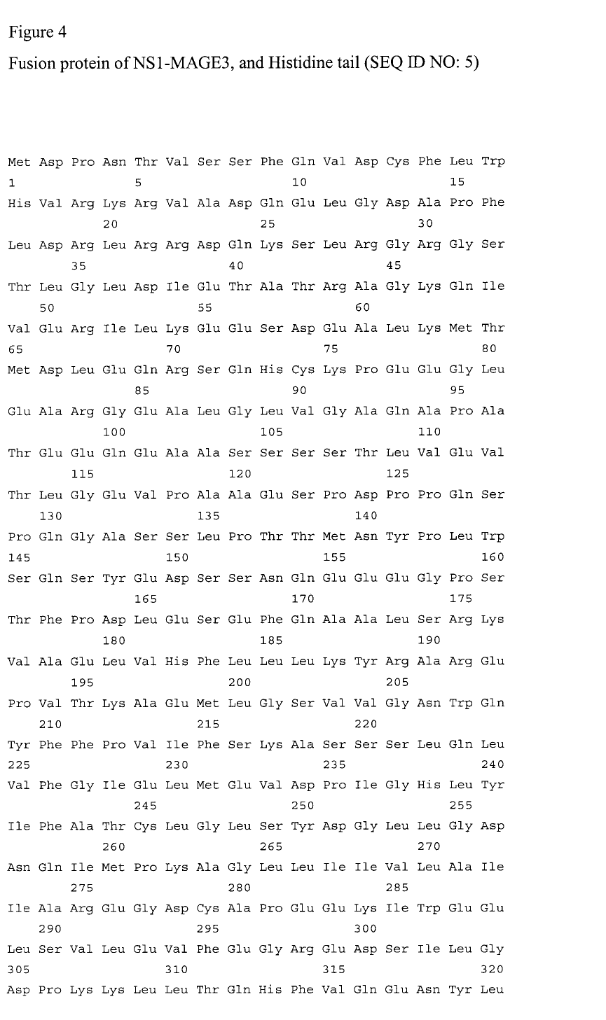

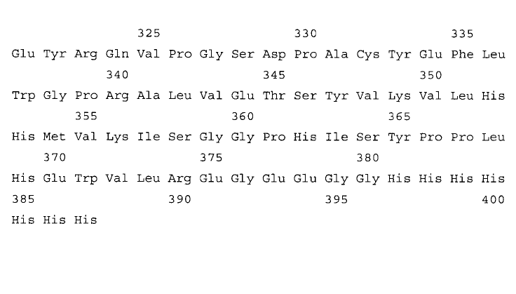

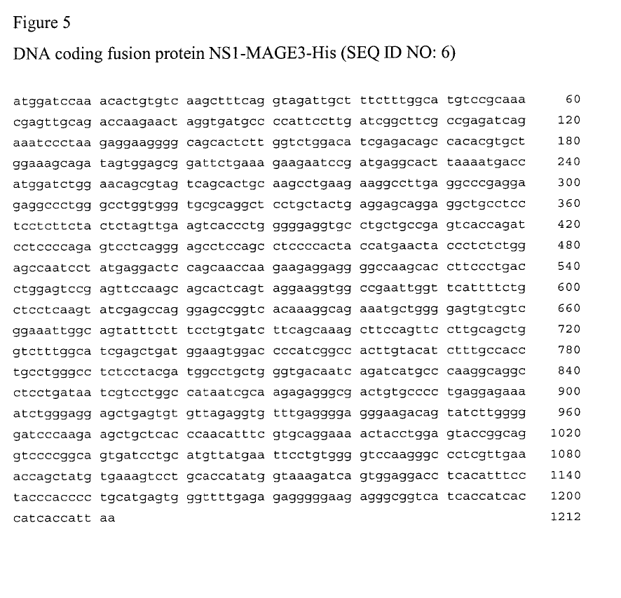

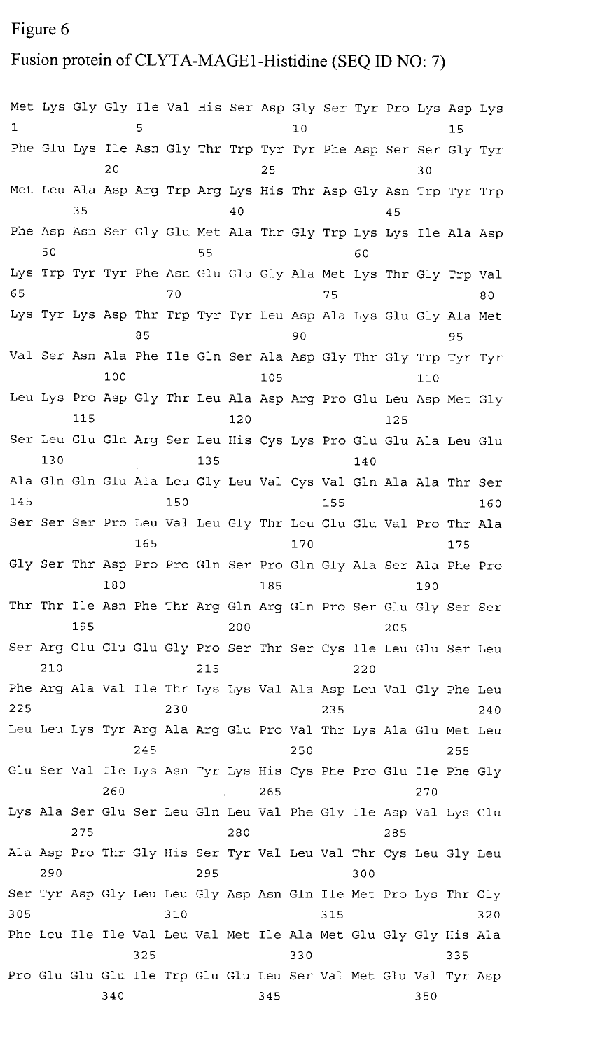

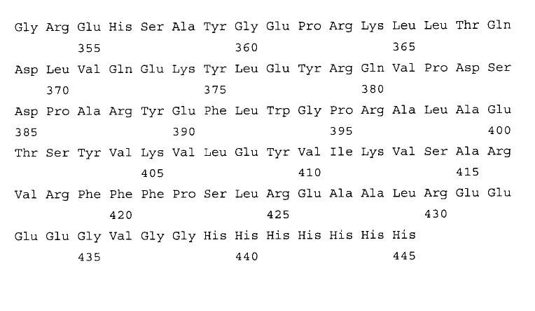

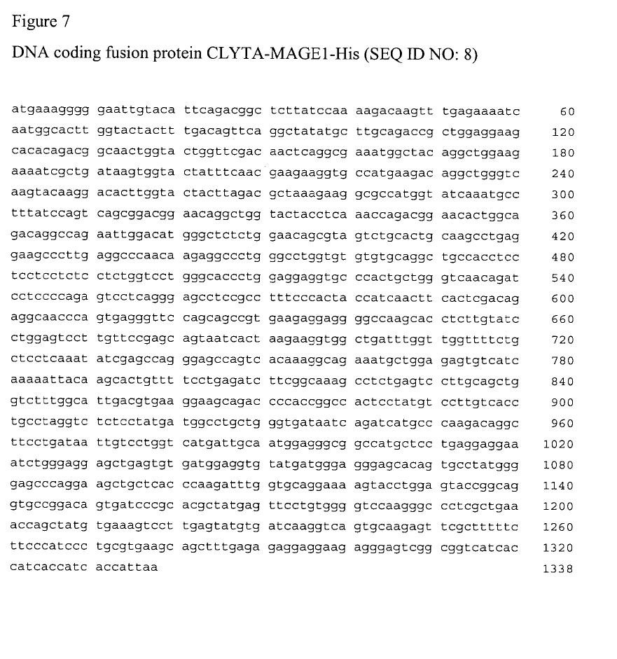

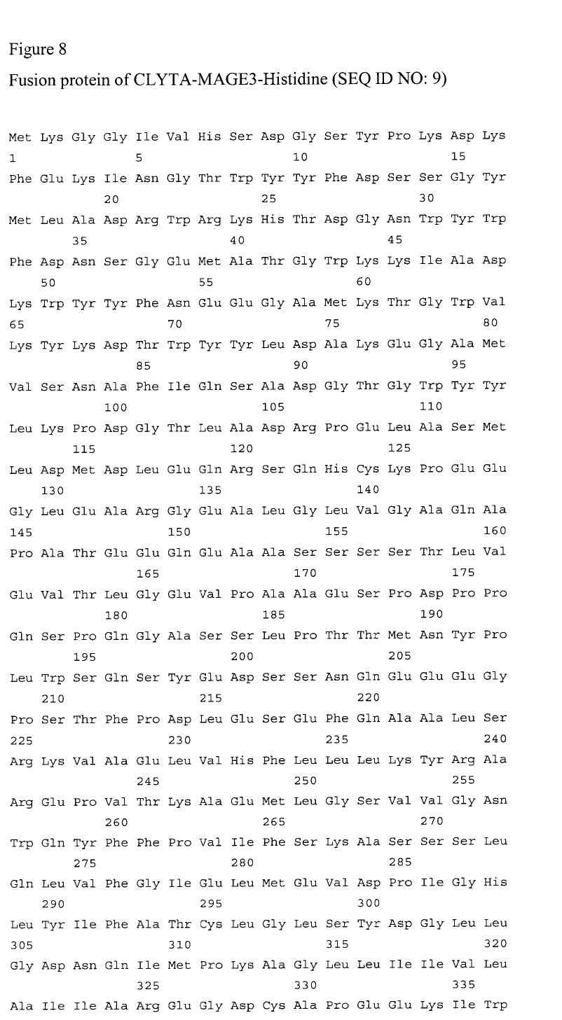

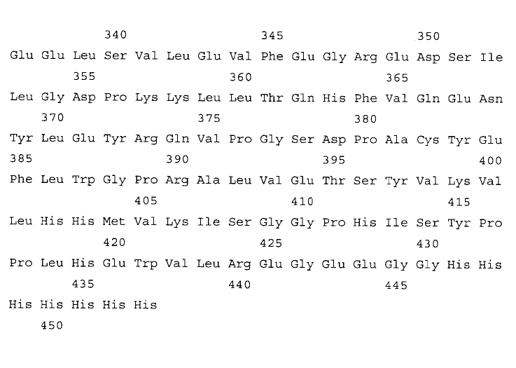

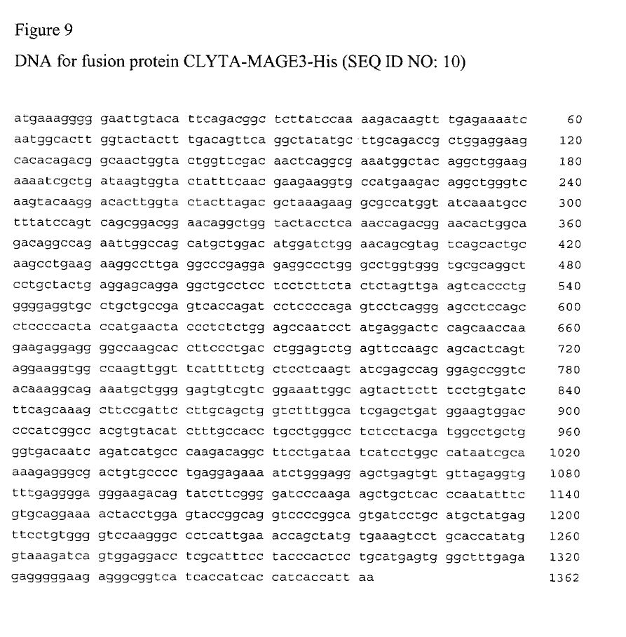

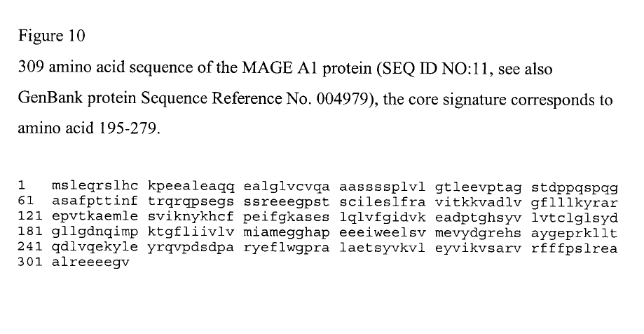

A fusion protein of Lipoprotein D fragment, MAGE 1 fragment, and histidine tail, for example as shown in FIGS. 2 and 3; a fusion protein of NS1-MAGE3, and histidine tail, for example as shown in FIGS. 4 and 5; a fusion protein of CLytA-MAGE1-histidine, for example as shown in FIGS. 6 and 7; a fusion protein of CLytA-MAGE3-histidine, for example as shown in FIGS. 8 and 9.

A further embodiment of the present invention comprises a nucleic acid molecule encoding the antigens as described herein. Such sequences may be inserted into a suitable expression vector and used for DNA/RNA vaccination or expressed in a suitable host. Microbial vectors expressing the nucleic acid may also be used. Such vectors include for example, poxvirus, adenovirus, alphavirus and listeria.

Conventional recombinant techniques for obtaining nucleic acid sequences, and production of expression vectors of are described in Maniatis et al., Molecular Cloning—A Laboratory Manual; Cold Spring Harbor, 1982-1989.

In particular, a process may comprise the steps of:

i) preparing a replicable or integrating expression vector capable, in a host cell, of expressing a DNA polymer comprising a nucleotide sequence that encodes the protein or an immunogenic derivative thereof;
ii) transforming a host cell with said vector;
iii) culturing said transformed host cell under conditions permitting expression of said DNA polymer to produce said protein; and
iv) recovering said protein.

The method as described herein may comprise a composition that may optionally contain one or more other tumour-associated antigens. For example other members belonging to the MAGE and LAGE families. In one embodiment, other tumour associated antigens that may also be included in combination with an antigen of the present invention include MAGE-A1, MAGE-A3, LAGE-1, GAGE-1 or Tyrosinase proteins.

The proteins of the present invention are provided either soluble in a liquid form or in a lyophilised form.

It is generally expected that each human dose will comprise 1 to 1000 μg of protein, and preferably 30-300 μg.

The method as described herein may comprise a composition further comprises an adjuvant, and/or immunostimulatory cytokine or chemokine.

When adjuvant is used in this specification in relation to a component of the immunotherapy it will generally relate to an agent which may boost an immune response to an antigen component of the immunotherapy.

Such adjuvants are well known in the art and can be administered in a separate formulation or may be a component of the formulation comprising the primary component of the immunotherapy.

Suitable adjuvants for use in the present invention are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminium salts such as aluminium hydroxide gel (alum) or aluminium phosphate; salts of calcium, iron or zinc; an insoluble suspension of acylated tyrosine; acylated sugars; cationically or anionically derivatised polysaccharides; polyphosphazenes; biodegradable microspheres; monophosphoryl lipid A and quil A. Cytokines, such as GM-CSF or interleukin-2, -7, or -12, and chemokines may also be used as adjuvants.

In formulations of the invention it may be desirable that an adjuvant induces an immune response predominantly of the Th1 type. High levels of Th1-type cytokines (e.g., IFN-γ, TNFα, IL-2 and IL-12) tend to favour the induction of cell mediated immune responses to an administered antigen. According to one embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Accordingly, suitable adjuvants that may be used to elicit a predominantly Th1-type response may include, for example, a combination of monophosphoryl lipid A, such as 3-de-O-acylated monophosphoryl lipid A (3D-MPL) together with an aluminium salt. 3D-MPL or other toll like receptor 4 (TLR4) ligands such as aminoalkyl glucosaminide phosphates as disclosed in WO9850399, WO0134617 and WO03065806 may also be used alone to generate a predominantly Th1-type response.

Other known adjuvants, which may preferentially induce a TH1 type immune response, include TLR9 antagonists such as unmethylated CpG containing oligonucleotides. The oligonucleotides are characterised in that the CpG dinucleotide is unmethylated. Such oligonucleotides are well known and are described in, for example WO 96/02555.

Suitable oligonucleotides include:

OLIGO 1(SEQ ID NO:12):
TCC ATG ACG TTC CTG ACG TT (CpG 1826)
OLIGO 2(SEQ ID NO:13):
TCT CCC AGC GTG CGC CAT (CpG 1758)
OLIGO 3(SEQ ID NO:14):
ACC GAT GAC GTC GCC GGT GAC GGC ACC ACG
OLIGO 4(SEQ ID NO:15):
TCG TCG TTT TGT CGT TTT GTC GTT (CpG 2006)
OLIGO 5(SEQ ID NO:16):
TCC ATG ACG TTC CTG ATG CT (CpG 1668)

CpG-containing oligonucleotides may also be used alone or in combination with other adjuvants. For example, one system that may be used in the present invention involves the combination of a CpG-containing oligonucleotide and a saponin derivative particularly the combination of CpG and QS21 as disclosed in WO 00/09159 and WO 00/62800. Another adjuvant formulation that may be used is QS21, 3D-MPL in combination with CpG or an equivalent thereof or CpR in an oil in water emulsion or as a liposomal formulation. Generally, the adjuvant for the immunotherapy will be a TLR 7, 8 or 9 agonist, such as a TLR 9 agonist.

The formulation may additionally comprise an oil in water emulsion and/or tocopherol.

Another suitable adjuvant is a saponin, for example QS21 (Aquila Biopharmaceuticals Inc., Framingham, Mass.), that may be used alone or in combination with other adjuvants. For example, an enhanced system involves the combination of a monophosphoryl lipid A and saponin derivative, such as the combination of QS21 and 3D-MPL as described in WO 94/00153, or a less reactogenic adjuvant where the QS21 is quenched with cholesterol, as described in WO 96/33739. Other suitable formulations comprise an oil-in-water emulsion and tocopherol. A further adjuvant formulation that may be used involving QS21, 3D-MPL and tocopherol in an oil-in-water emulsion is described in WO 95/17210.

In another embodiment, the adjuvants may be formulated in a liposomal composition. The amount of 3 D MPL used is generally small, but depending on the vaccine formulation may be in the region of 1-1000 μg per dose, preferably 1-500 μg per dose, and more preferably between 1 to 100 μg per dose.

The amount of CpG or immunostimulatory oligonucleotides in the adjuvants or vaccines of the present invention is generally small, but depending on the vaccine formulation may be in the region of 1-1000 μg per dose, preferably 1-500 μg per dose, and more preferably between 1 to 100 μg per dose.

The amount of saponin for use in the adjuvants of the present invention may be in the region of 1-1000 μg per dose, preferably 1-500 μg per dose, more preferably 1-250 μg per dose, and most preferably between 1 to 100 μg per dose.

Generally, it is expected that each human dose will comprise 0.1-1000 μg of antigen, preferably 0.1-500 μg, preferably 0.1-100 μg, most preferably 0.1 to 50 μg. An optimal amount for a particular vaccine can be ascertained by standard studies involving observation of appropriate immune responses in vaccinated subjects. Following an initial vaccination, subjects may receive one or several booster immunisation adequately spaced.

Other suitable adjuvants include Montanide™ ISA 720 (Seppic, France), SAF (Chiron, Calif., United States), ISCOMS (CSL), MF-59 (Chiron), Ribi Detox, RC-529 (GSK, Hamilton, Mont.) and other aminoalkyl glucosaminide 4-phosphates (AGPs).

Accordingly there is provided an immunogenic composition for use in the method of the present invention comprising an antigen as disclosed herein and an adjuvant, wherein the adjuvant comprises one or more of 3D-MPL, QS21, a CpG oligonucleotide, a polyethylene ether or ester or a combination of two or more of these adjuvants. The antigen within the immunogenic composition may be presented in an oil in water or a water in oil emulsion vehicle or in a liposomal formulation.

In one embodiment, the adjuvant may comprise one or more of 3D-MPL, QS21 and an immunostimulatory CpG oligonucleotide. In an embodiment all three immunostimulants are present. In another embodiment 3D MPL and Qs21 are presented in an oil in water emulsion, and in the absence of a CpG oligonucleotide.

A composition as described herein for use in the method of the present invention may additionally comprise a pharmaceutically acceptable excipient.

### Method of Treatment Schedules

### Adjuvant Setting

In one embodiment of the present invention, there is provided a method of treatment schedule for use in the treatment of MAGE-expressing cancers, including, but not limited to: melanoma; breast cancer; bladder cancer, including transitional cell carcinoma; lung cancer including non-small cell lung cancer (NSCLC); head and neck cancer, including: squamous cell carcinoma and oesophagus carcinoma; colon carcinoma; multiple myeloma, in an adjuvant setting, comprising: administration of a MAGE antigen that matches the MAGE antigen expressed by the tumor as described herein according to the following schedules.

By “adjuvant setting” is meant that a patient has had treatment, for example surgery, to remove all or substantially all detectable cancer tissue from the body. The patient may be apparently free of disease. In one embodiment, the patient may have had one or more, or all, of the following procedures to remove cancer tissue: surgery; radiotherapy; and chemotherapy. As will be known to those of ordinary skill in the art, removal of a primary tumor may still leave micrometastases in the patient's body. As used herein, micrometastases refer to small numbers of cancer cells that have spread from the primary tumor to other parts of the body, but are too few in number to be identified in available screening or diagnostic tests. “Complete surgical resection” does not encompass removal of all micrometastases.

Administration of antigen at three week intervals for the first 5 to 8 vaccinations, followed at 3 month intervals for the next 8, 9 or more vaccinations.

The antigen may be administered at the exact time frame indicated, or the antigen may be given 1, 2, 3 or 4 days before or after the exact interval, as required or as practical. An example of this schedule is shown in the table below:

Induction: 5 vaccinations at intervals of 3 weeks (Weeks 0, 6, 9, 12)
Maintenance: 9 vaccinations at intervals of 3 months

### Active Disease

In a further embodiment of the present invention, there is provided a method of treatment schedule for use in MAGE-expressing cancers, including, but not limited to: melanoma; breast cancer; bladder cancer, including transitional cell carcinoma; lung cancer including non-small cell lung cancer (NSCLC); head and neck cancer, including: squamous cell carcinoma; and oesophagus carcinoma; colon carcinoma; multiple myeloma; in active or unresectable disease, comprising: administration of an antigen as described herein at two or three week intervals for the first six months to one year of treatment. A schedule may comprise the following pattern of injections: the antigen may be given at 2 week intervals for the first 4 to 10 vaccinations, followed by 3 week intervals for the next 4 to 10 vaccinations, then at 6 week intervals for the next 3 to 7 vaccinations. Long term treatment may then continue with vaccinations at 3 month intervals for 3 to 5 vaccinations, followed by 6 month intervals for the next 3 to 5 vaccinations.

The antigen may be administered at the exact time frame indicated, or the antigen may be given 1, 2, 3 or 4 days before or after the exact interval, as required or as practical.

An example of this schedule is shown in the table below:

Cycle 1: 6 vaccinations at intervals of 2 weeks (Weeks 1, 3, 5, 7, 9, 11)
Cycle 2: 6 vaccinations at intervals of 3 weeks (Weeks 15, 18, 21, 24, 27, 30)
Cycle 3: 4 vaccinations at intervals of 6 weeks (Weeks 34, 40, 46, 52)
Long Term 4 vaccinations at intervals of 3 months
Treatment: 4 vaccinations at intervals of 6 months

For both the adjuvant setting and active setting treatment regimes described above, additional vaccinations may be given after treatment, as required.

It is generally expected that each human dose will comprise 1 to 1000 μg of protein, and preferably 30-300 μg. In one embodiment, each human dose will comprise 300 μg of protein.

The schedules for active treatment of disease are generally more aggressive. However, in one embodiment of the present invention the “active disease” setting schedule as described herein may be used for patients in the “adjuvant setting”. For example, the schedule for active disease may be used with WT-1 antigen specific cancer immunotherapy, in adjuvant setting WT1-positive leukaemia.

### EXAMPLES

### Example 1

### NSCLC (Adjuvant Setting)

### Example 1.1 Methods

Patients were enrolled with completely resected, MAGE-A3 positive (assessed by quantitative RT-PCR) NSCLC at stage IB or II were 2:1, double-blind, randomly assigned to post-operative MAGE-3 vaccination or placebo. The study treatment is given as an adjuvant treatment and is composed of 300 μg of recombinant Prot.D MAGE-A3/His formulated in adjuvant AS02B, 0.5 ml) or matching placebo (sucrose dissolved in an oil-in-water emulsion, 0.5 ml) was administered by intramuscular injection. Adjuvant AS02B comprises MPL and QS21 in an oil in water emulsion and may be prepared as follows: Preparation of oil in water emulsion followed the protocol as set forth in WO 95/17210. The emulsion contains: 5% Squalene 5% tocopherol 2.0% tween 80; the particle size is 180 nm.

### Preparation of Oil in Water Emulsion (2 Fold Concentrated)

Tween 80 was dissolved in phosphate buffered saline (PBS) to give a 2% solution in the PBS. To provide 100 ml two fold concentrate emulsion 5 g of DL alpha tocopherol and 5 ml of squalene were vortexed until mixed thoroughly. 90 ml of PBS/Tween solution was added and mixed thoroughly. The resulting emulsion was then passed through a syringe and finally microfluidised by using an M110S microfluidics machine. The resulting oil droplets have a size of approximately 180 nm.

To the Sterile bulk emulsion is added to PBS to reach a final concentration of 250 or 500 μl of emulsion per ml (v/v). 3 D-MPL is then added to reach a final concentration of 100 μg/ml and QS21 is then added to reach a final concentration of 100 μg per ml. Between each addition of component, the intermediate product was stirred for 5 minutes. Once antigen is added the pH can be adjusted.

For inclusion in the described clinical trial, patients must have had complete surgical resection of their tumours. They must not have received anti-cancer neo-adjuvant therapy, may not be candidates for radiotherapy, and must be free of any asymptomatic metastasis, as shown by a negative baseline computer tomogram (CT scan) of the brain, chest and upper abdomen and optionally a radionuclide bone scan.

Vaccination was started <6 weeks after surgery, with 5 vaccinations at 3-week intervals, followed by 8 vaccinations every 3 months. Randomization was stratified for stage (IB vs II), histology (squamous vs other), and lymph node procedure (sampling vs dissection). Other anti-cancer adjuvant therapy was not allowed in the trial.

Primary endpoint was time-to-recurrence, other endpoints were recurrence rates at different times, and survival rate. The sample size of 180 patients (120 active, 60 placebo) achieved 48% power (α=10%) to detect a 10% difference, assuming a 40% recurrence rate at month 30 in the placebo group (log rank test). A thorough interim efficacy analysis by an independent statistician (with all investigators and the sponsor blinded) is planned at 18 months after completion of recruitment.

### Example 1.2 Results

About 35% of 1089 biopsies obtained from 59 treatment centres were MAGE-3 positive. In two years, 182 patients were randomized (121 stage IB, 61 stage II). Data collection for the interim analysis was initiated on Dec. 19, 2005. Median follow-up was 21 months, with 62 recurrences recorded. Overall, treatment was well tolerated, with high protocol compliance.

### Example 1.3 Conclusions

This study confirmed the frequency of expression of MAGE-3 antigen in early NSCLC (35%), and demonstrated good tolerability of postoperative MAGE-3 vaccination. Interim analysis of the randomised Phase II trial show reasonably reliable results overall, with a risk reduction for disease-free survival of around 33% in the group receiving the MAGE-A3 recombinant protein combined with AS02B immunological adjuvant (MPL, QS21 in an oil in water emulsion). The risk reduction did not reach statistical significance (p=0.121). However, this is not surprising because the trial was not designed to detect such a difference with statistical significance.

### Example 2

### Melanoma Cancer

### Example 2.1—Methods

In an on-going Phase II clinical trial, groups of patients receive MAGE-3-Protein D-His antigen combined with one of two different immunological adjuvants: in group 1 the antigen is combined with AS02B (MPL, QS21 in an oil in water emulsion); in group 2 the antigen is combined with AS15 (Liposomal formulation of MPL combined with QS21 and CpG7909).

AS02B may be prepared as described above. AS15 may be prepared following the protocol shown below:

Sterile bulk CpG was added to PBS solution to reach a final concentration of 100 μg/ml. PBS composition was Na₂HPO₄: 9 mM; KH2PO4: 48 mM; NaCl: 100 mM pH 6.1. Antigen is then added to reach. Finally, QS21 and 3D-MPL were added as a premix of sterile bulk small unilamelar vesicles (SUV), containing 3D-MPL and QS21 (referred as DQMPL) to reach final 3D-MPL and QS21 concentrations of 10 μg/ml.

A mixture of lipid (such as phosphatidylcholine either from egg-yolk or synthetic) and cholesterol and 3D-MPL in organic solvent, was dried down under vacuum (or alternatively under a stream of inert gas). An aqueous solution (such as phosphate buffered saline) was then added, and the vessel agitated until all the lipid was in suspension. This suspension was then microfluidised until the liposome size was reduced to about 100 nm, and then sterile filtered through a 0.2 μm filter. Extrusion or sonication could replace this step.

Typically the cholesterol:phosphatidylcholine ratio was 1:4 (w/w), and the aqueous solution was added to give a final cholesterol concentration of 5 to 50 mg/ml.

The liposomes have a defined size of 100 nm and are referred to as SUV (for small unilamelar vesicles). The liposomes by themselves are stable over time and have no fusogenic capacity. Sterile bulk of SUV was added to PBS to reach a final concentration of 10, 20 or 100 μg/ml of 3D-MPL. PBS composition was Na2HPO4: 9 mM; KH2PO4: 48 mM; NaCl: 100 mM pH 6.1. QS21 in aqueous solution was added to the SUV. This mixture is referred as DQMPLin.

The MAGE-3-AS15 composition for use in the above mentioned melanoma trial (MAGE008) is prepared by combining 3 vials: 1 lyophilised MAGE3 protein 300 μg; 2. liquid CpG (500 μg); and 3. Liquid AS01B (50 μgQS21-50 μg MPL)

MAGE3 AS15 can also be given as a 2 vials approach when the MAGE3 protein and the CPG are co-lyophylized and resuspended in liquid AS01B. Preparation of AS15 for use in the clinical trial MAGE 008 comprised combining: a mixture of equal volumes of the adjuvants AS7A and AS 1B. AS7A contains 500 μg of CpG7909 in 500 μl. AS1B contains 50 μg QS21 and 50 μg MPL made up to 500 μl with a suspension of liposomes.

The objectives of this trial were to discriminate between the adjuvants in terms of safety profile, clinical response and immunological response. This trial is open, randomized, two-arm (AS02B vs. AS15) and included 68 patients in total. CpG7909 is obtainable from Coley Pharmaceuticals.

In this trial, the antigen is administered to patients with progressive metastatic melanoma with regional or distant skin and/or lymph-node lesions (unresectable stage III and stage IV M1a). The expression of the MAGE-A3 gene by the tumor was assessed by quantitative PCR. The selected patients did not receive previous treatment for melanoma (MAGE-A3 antigen is given as first-line treatment) and had no visceral disease.

### Stratification Factors

Patients are being included according to three stratification factors:
stage III in transit vs. stage III unresectable vs. stage IV M1a,
presence or absence of a lesion ≧20 mm,
Medical treatment center.

### Objectives

The primary objectives of this study are:
The safety of the injections
The objective clinical response

The secondary endpoints are:
The rate of stable disease,
The rate of mixed response (tumor regression but no objective response to describe any clinical activity)
The rate of immune response (analyzed quantitatively and qualitatively).

### 7.1 Objective Tumor Response —RECIST Criteria—for the MAGE 008 Study

For the clinical studies referred to herein, objective tumor response was measured according to the RECIST criteria as a primary endpoint of this study.

Response criteria are essentially based on a set of measurable lesions identified at baseline as target lesions, and followed until disease progression.

The following paragraphs are a quick reference to the RECIST criteria (Ref. 11) as appropriate to this study. The complete criteria are available at:
http://www3.oup.co.uk/jnci/extra/920205.pdf.

### 7.1.1. Measurability of Tumor Lesions at Baseline

### 7.1.1.1. Definitions

Measurable disease: the presence of at least one measurable lesion. If the measurable disease is restricted to a solitary lesion, its neoplasic nature should be confirmed by cytology/histology.
Measurable lesions: lesions that can be accurately measured in at least one dimension with longest diameter ≧20 mm. With spiral CT scan, the lesion must be ≧10 mm in at least one dimension.
Non-measurable lesions: all other lesions, including small lesions (longest diameter <20 mm with conventional techniques or <10 mm with spiral CT scan) and other non-measurable lesions. These include: bone lesions, leptomeningeal disease; ascites; pleural/pericardial effusion; inflammatory breast disease; lymphangitis cutis/pulmonis; abdominal masses that are not confirmed and followed by imaging techniques; and cystic lesions.

All measurements should be recorded in metric notation by use of a ruler or calipers. All baseline evaluations should be performed as closely as possible to the beginning of treatment and never more than 4 weeks before the beginning of the treatment.

### 7.1.1.2. Methods of Measurements

For the clinical trial referred to herein, the same method of assessment and the same technique should be used to characterize each identified and reported lesion at baseline and during follow-up.
Clinically detected lesions will only be considered measurable when they are superficial (e.g. skin nodules, palpable lymph nodes). For the case of skin lesions, documentation by color photography—including a ruler to estimate the size of a lesion—is required.
CT and MRI are the best currently available and reproducible methods to measure target lesions selected for response assessment. Conventional CT and MRI should be performed with contiguous cuts of 10 mm or less in slice thickness. Spiral CT should be performed using a 5 mm contiguous reconstruction algorithm; this specification applies to the tumors of the chest, abdomen and pelvis while head and neck tumors and those of the extremities usually require specific protocols.
Ultrasound (US) should not be used to measure tumor lesions that are clinically not easily accessible. It may be used as a possible alternative to clinical measurements of superficial palpable nodes, subcutaneous lesions. US might also be useful to confirm the complete disappearance of superficial lesions usually assessed by clinical examination. US should preferentially be performed by the same investigator.

### 7.1.2. Tumor Response Evaluation—for the Clinical Trial Referred to Herein

### 7.1.2.1 Baseline Documentation of “Target” and “Non-Target” Lesions

All measurable lesions up to a maximum of 5 lesions per organ and 10 lesions in total, representative of all involved organs, should be identified as target lesions and will be recorded and measured at baseline.

Target lesions should be selected on the basis of their size (those with the longest diameter) and their suitability for accurate repetitive measurements (either by imaging techniques or clinically).

A sum of the longest diameter (LD) for all target lesions will be calculated and reported as the baseline sum LD. The baseline sum LD will be used as reference by which to characterize the objective tumor response.

All other lesions (or sites of disease) should be identified as non-target lesions and should also be recorded and measured at baseline. Measurements are not required but the presence or absence of each should be noted throughout follow-up.

### 7.1.2.2. Response Criteria

### Evaluation of Target Lesions

Complete response (CR)—Disappearance of all target lesions.

Partial response (PR)—At least a 30% decrease in the sum of LD of target lesions taking as reference the baseline sum LD.

Stable disease (SD)—Neither sufficient shrinkage to qualify for PR nor sufficient increase to qualify for PD taking as references the smallest sum LD since the treatment started.

Progressive disease (PD)—At least a 20% increase in sum of LD of target lesions taking as references the smallest sum LD recorded since the treatment started OR the appearance of one or more new lesions OR both of these.

Complete response (CR)—Disappearance of all non-target lesions.

Incomplete response/stable disease—Persistence of one or more of non-target lesion(s).

Progressive disease (PD)—Appearance of one or more new lesions and/or unequivocal progression of existing non-target lesions (*).

(*) Although a clear progression of “non-target” lesions only is exceptional, in such circumstances, the opinion of the treating physician should prevail.

### Evaluation of Best Overall Response—Definition Used for the Clinical Trial Referred to Herein

The best overall response is the best response recorded from the start of the treatment until disease progression/recurrence (taking as reference for progressive disease the smallest measurements recorded since the treatment started). In general the patient's best response assignment will depend on the achievement of both measurement and confirmation criteria.

Patients with a global deterioration of health status requiring discontinuation of treatment without objective evidence of disease progression at that time should be reported as “symptomatic deterioration”. Every effort should be made to document the objective progression even after discontinuation of treatment.

In some circumstances it may be difficult to distinguish residual disease from normal tissue. When the evaluation of complete response depends upon this determination, it is recommended that the residual lesion be investigated (fine needle aspirate/biopsy) before confirming the complete response status.

### Schedule of Immunization

The immunization scheme comprises an active phase and a long-term treatment phase. The active phase comprises three cycles over a period of 54 weeks. During the induction (cycle 1), six injections are given every two weeks. For maintenance, six injections are given every three weeks in cycle 2 and four injections are given every six weeks in cycle 3. The long-term treatment comprises four injections every three months and then four injections every six months. Overall, 24 injections may be given over a period of about 4 years.

### Study Status

From a total of 133 screened melanoma patients, 121 tumors were tested for MAGE-A3 expression and 70 (58%) of these were MAGE-A3 positive. Of the patients with MAGE-A3 positive tumors, 58 have been randomized so far, with the aim of obtaining 68 randomized patients in total.

### Example 2.2 Conclusion

We can conclude that there appears to be a difference between responses in the two arms: better efficacy of MAGE-A3 antigen in AS15 adjuvant is suggested by the reduced number of patients with progressive disease. Further analysis will occur after completion of the study.

What is claimed is:

1. A method of adjuvant treatment of a human patient having MAGE A3 positive cancer, comprising administering to the patient a composition comprising a tumour associated MAGE A3 antigen, in which the composition is administered at three week intervals for the first 5 to 8 vaccinations, followed by at least eight additional vaccinations at 3 month intervals.

2. (canceled)

3. A method according to claim 1 in which the composition administration schedule comprises: (a) an induction phase consisting of five vaccinations at intervals of three weeks, and (b) a subsequent maintenance phase comprising at least nine vaccinations at intervals of three months.

4. A method of treating a human patient having active MAGE A3 positive cancer, comprising administering to the patient a composition comprising a tumour associated MAGE A3 antigen, in which the composition is administered at two or three week intervals for the first six months to one year of treatment.

5.-6. (canceled)

7. A method according to claim 1 in which the composition is further administered at 3 month intervals for 3 to 5 vaccinations, followed by 6 month intervals for the next 3 to 5 vaccinations.

8. A method according to claim 4 in which the composition is administered according to the schedule: six vaccinations at intervals of two weeks; six vaccinations at intervals of three weeks; four vaccinations at intervals of six weeks; four vaccinations at intervals of three months; and four vaccinations at intervals of six months.

9.-10. (canceled)

11. The method of claim 1, in which the antigen is linked to an immunological fusion or expression enhancer partner.

12. The method according to claim 11 in which the fusion partner is selected from the group consisting of: protein D or a fragment thereof from Haemophilus influenzae B; NS1 protein from influenza or a fragment thereof; and LytA from Streptococcus pneumoniae or a fragment thereof.

13. The method according to claim 11 in which the fusion partner is the lipidated form of protein D or fragment thereof.

14. The method according to claim 1, in which the antigen further comprises an affinity tag.

15. The method according to claim 1, in which the antigen further comprises a derivatised free thiol.

16. The method according to claim 15 in which the derivatised free thiol is a carboxyamide or carboxymethylated derivative.

17. The method according to claim 30 in which the nucleotide sequence is comprised within a vector.

18. (canceled)

19. The method according to claim 1 in which the composition further comprises an adjuvant, immunostimulatory cytokine or chemokine.

20. The method according to claim 19 in which the adjuvant comprises one or more of 3D-MPL, QS21 and a CpG immunostimulatory oligonucleotide.

21.-22. (canceled)

23. The method according to claim 1, in which the MAGE A3 positive cancer is stage Ib, II or IIIa non small cell lung carcinoma (NSCLC) and said composition comprises (a) an antigen comprising SEQ ID NO:2 and (b) an adjuvant.

24.-25. (canceled)

26. The method according to claim 1 in which the patient has had surgical resection of all detectable cancer tissue.

27. The method according to claim 4, in which the cancer is selected from melanoma; breast cancer; liver cancer; bladder cancer including transitional cell carcinoma; lung cancer including non-small cell lung cancer (NSCLC); head and neck cancer including squamous cell carcinoma and oesophagus carcinoma; colon carcinoma; seminoma; and multiple myeloma.

28. A method according to claim 4, where said MAGE positive cancer is MAGE A3 positive melanoma and said composition comprises (a) an antigen comprising SEQ ID NO:2 and (b) an adjuvant comprising a CpG immunostimulatory oligonucleotide.

29. A method of adjuvant treatment of a human patient having MAGE positive cancer, comprising administering to the patient a composition comprising a nucleotide sequence encoding a tumour associated MAGE antigen, in which the composition is administered at three week intervals for the first 5 to 8 vaccinations, followed by at least eight additional vaccinations at 3 month intervals.
